(19) United States
(12) Patent Application Publication
(10) Pub. No.: US 2025/0152364 A1
(43) Pub. Date: May 15, 2025

(54) EXPANDABLE DEVICE TO IMPROVE FIXATION OF ORTHOPAEDIC IMPLANTS

(71) Applicant: Christopher TRAVERS, Philadelphia, PA (US)

(72) Inventor: Christopher TRAVERS, Philadelphia, PA (US)

(21) Appl. No.: 18/942,678

(22) Filed: Nov. 9, 2024

Related U.S. Application Data
(60) Provisional application No. 63/597,838, filed on Nov. 10, 2023.

(51) Int. Cl. A61F 2/28; A61F 2/30

(52) U.S. Cl. CPC A61F 2/2846; A61F 2002/30579; A61F 2220/0025

(57) ABSTRACT

An adapter for receiving an implant includes at least one rigid segment and at least one flexible segment connected to the at least one rigid segment. The at least one rigid segment and the at least one flexible segment together form a body. The at least one rigid segment may be made from a material encouraging growth of bone thereinto. The body defines an interior and an exterior. The at least one rigid segment and the at least one flexible segment bias the body to expand outwardly from the interior. The body is adapted to be inserted into a bone.

### CROSS-REFERENCE TO RELATED APPLICATION(S)

This Non-Provisional United States Patent Application relies for priority on U.S. Provisional Patent Application Ser. No. 63/597,838, filed on Nov. 10, 2024, the entire content of which is incorporated herein by reference.

### FIELD OF THE INVENTION

The present invention encompasses, inter alia, a device that is implantable in a long bone to improve fixation of an orthopaedic implant in the long bone.

### DESCRIPTION OF THE RELATED ART

In the prior art, when inserting an orthopaedic implant into a long bone, it is occasionally customary to first place an insert into the long bone. The insert may be press fit or cemented into the long bone. The insert is installed so that the orthopaedic implant has a suitable connection to the long bone.

In one example, it is customary to install an insert into the proximal end of a human tibia when installing an artificial knee in a patient. So that the artificial knee is provided with a strong connection to the proximal end of the tibia, an insert is first placed into the central channel of the tibia, at the proximal end of the tibia. The insert may be cemented in place.

The insert is constructed to bond strongly to the proximal end of the tibia. The replacement knee is then installed in the insert. In this manner the artificial knee is suitably connected to the proximal end of the patient's tibia.

As should be apparent to those skilled in the art, the central channel in the proximal end of a patient's tibia does not have the same dimensions from patient to patient. As a result, sometimes the insert is too big for a particular surgery. In other cases, the insert may be slightly undersized.

In the prior art, it is customary to have multiple sizes for the insert. As such, the surgeon may select an appropriate size for each patient.

As should be apparent to those skilled in the art, this approach to sizing inserts is suitable, but it leaves room for improvement.

The prior art fails to provide a practical solution to this problem.

### SUMMARY OF THE INVENTION

The present invention seeks to address one or more deficiencies in the prior art.

Specifically, the present invention provides an adapter for receiving an implant. The adapter includes at least one rigid segment and at least one flexible segment connected to the at least one rigid segment. The at least one rigid segment and the at least one flexible segment together form a body. The at least one rigid segment is contemplated to be made from a material encouraging growth of bone thereinto. The body defines an interior and an exterior. The at least one rigid segment and the at least one flexible segment bias the body to expand outwardly from the interior. The body is adapted to be inserted into a bone.

In one embodiment, it is contemplated that the at least one rigid segment is made from at least one of sintered titanium, a porous metal alloy, a porous ceramic material, a polymer, and a biocompatible material.

Still further, the at least one flexible segment may be made from titanium, a metal alloy, a shape metal alloy, a ceramic, a polymer, and a biocompatible material.

In another embodiment, the body is frusto-conically shaped.

Alternatively, the body may be cylindrically shaped.

Still further, the body may define a mesh.

In an additional embodiment, the at least one flexible segment is a spring.

As a variation, the at least one flexible segment may be a scissor element.

It is contemplated that the at least one flexible segment is C-shaped.

In another embodiment, the at least one rigid segment may include at least two rigid segments separated from one another by gaps.

It is also contemplated that the at least one flexible segment may include a plurality of flexible segments disposed in the gaps, connecting the at least two rigid segments together.

Where the at least one flexible element is C-shaped, the C-shaped flexible segment may define a keyway, with a key insertable into the keyway.

It is contemplated that the at least one flexible segment may form a mesh and the at least one rigid segment may be disposed on the at least one flexible segment.

It is also contemplated that the at least one rigid segment and the at least one flexible segment together form the mesh.

Additional advantages and features of the present invention will be made apparent by the discussion presented hereinbelow.

### BRIEF DESCRIPTION OF THE DRAWINGS

The present invention will now be described in connection with the drawings appended hereto, in which:

FIG. 1 is a graphical illustration providing a general overview of the prior art;

FIG. 2 is a graphical, perspective illustration of a first embodiment of an adapter according to the present invention;

FIG. 3 is a graphical, top view of a second embodiment of the adapter of the present invention;

FIG. 4 is a graphical, perspective illustration of a third embodiment of an adapter according to the present invention;

FIG. 5 is a graphical, top view of a fourth embodiment of the adapter of the present invention;

FIG. 6 is a graphical, perspective illustration of a fifth embodiment of an adapter according to the present invention;

FIG. 7 is a graphical, top view of a sixth embodiment of the adapter of the present invention;

FIG. 8 is a graphical, perspective illustration of a seventh embodiment of an adapter according to the present invention;

FIG. 9 is a graphical, perspective illustration of an eighth embodiment of an adapter according to the present invention; and

FIG. 10 is a graphical, perspective illustration of a ninth embodiment of an adapter according to the present invention.

### DETAILED DESCRIPTION OF EMBODIMENT(S) OF THE INVENTION

The present invention will now be described in connection with several examples and embodiments. The present invention should not be understood to be limited solely to the examples and embodiments discussed. To the contrary, the discussion of selected examples and embodiments is intended to underscore the breadth and scope of the present invention, without limitation. As should be apparent to those skilled in the art, variations and equivalents of the described examples and embodiments may be employed without departing from the scope of the present invention.

In addition, aspects of the present invention will be discussed in connection with specific materials and/or components. Those materials and/or components are not intended to limit the scope of the present invention. As should be apparent to those skilled in the art, alternative materials and/or components may be employed without departing from the scope of the present invention.

In the illustrations appended hereto, for convenience and brevity, the same reference numbers are used to refer to like features in the various examples and embodiments of the present invention. The use of the same reference numbers for the same or similar structures and features is not intended to convey that each element with the same reference number is identical to all other elements with the same reference number. To the contrary, the elements may vary from one embodiment to another without departing from the scope of the present invention.

Still further, in the discussion that follows, the terms “first,” “second,” “third,” etc., may be used to refer to like elements. These terms are employed to distinguish like elements from similar examples of the same elements. For example, one fastener may be designated as a “first” fastener to differentiate that fastener from another fastener, which may be designated as a “second fastener.” The terms “first,” “second,” “third,” are not intended to convey any particular hierarchy between the elements so designated.

It is noted that the use of “first,” “second,” and “third,” etc., is intended to follow common grammatical convention. As such, while a component may be designated as “first” in one instance, that same component may be referred to as “second, “third,” etc., in a separate instance. The use of “first,” “second,” and “third,” etc., therefore, is not intended to limit the present invention.

In the paragraphs that follow, the present invention will be described in connection with knee replacement surgery. It is noted, however, that the present invention is not limited to knee replacement surgery. To the contrary, the present invention is contemplated to be useable in any instance where an orthopaedic implant is positioned in a bone, such a long bone, in the human body. For example, the present invention may be used in a human femur.

Still further, while the present invention is described in connection with orthopaedic implants for humans, the present invention is applicable in other animal species as well. In other words, the present invention should not be limited solely to human orthopaedic implants.

As should be apparent to those skilled in the art, fixation of orthopaedic implants to a patient's skeleton requires intimate contact between bone and one or more implant/cement surfaces. By ensuring an intimate fit, preferably with a broad surface area for contact, an implant achieves axial, rotational, and translational stability allowing the device to function as intended. This intimate fit is of particular important in situations where bone stock is poor or is diminished by a prior surgery.

Using revision total knee replacements as an example, prior implant failure and the need to remove a previous implant from the bone often lead to defects of the joint surface, metaphysis, and diaphysis. With this, there is less bone mass and surface area for the implant to gain purchase.

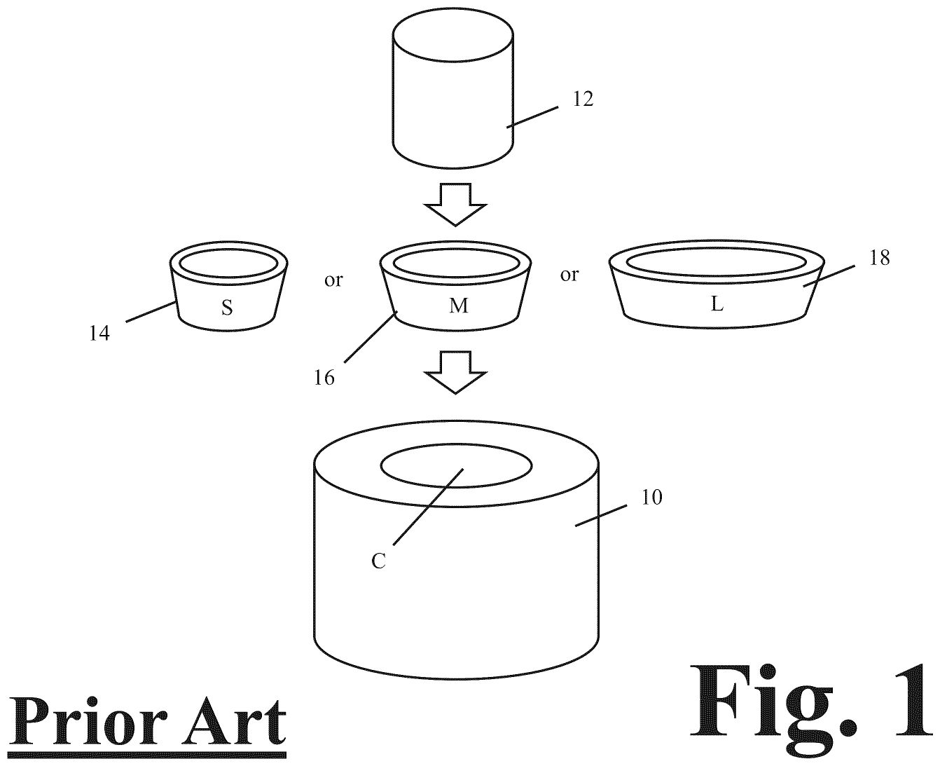

As discussed in greater detail in connection with FIG. 1, below, one modern solution to this problem is to use a device (sometimes termed a “cone” or a “sleeve”) that is impacted into the defect to bolster the surrounding bone. While designs vary from one manufacturer to another, the devices are typically rigid, monobloc, hollow constructions that are impacted into the bone allowing an implant to be passed through and affixed to their hollow, pipe-like interior. These cones or sleeves come in different shapes and sizes and may be thought of as a ring or adapter that helps mate the implant to the bone in a setting of a bony defect or a void that is larger than the implant.

One problem with this approach is that the defects encountered in revision surgery (e.g., a second knee replacement) and/or the patient's bony anatomy are highly variable. With large, uneven defects or a smaller bony anatomy, the preparation and impaction of these rigid devices can be difficult, if not impossible. Moreover, the insertion of these devices potentially poses a risk of bone fracture from the force of impaction. As a result, while these stabilizing devices can have a positive effect on implant fixation, they are not always useable and can place a patient at increased risk of bone fracture.

FIG. 1 is a graphical illustration of the problem identified above with respect to the prior art.

FIG. 1 graphically illustrates the proximal end of a tibia 10.

To secure an implant 12 into the proximal end of the tibia 10, a surgeon selects an adapter 14, 16, 18 with a suitable size. Here, three adapters 14, 16, 18 are illustrated. As indicated in FIG. 1, the adapters 14, 16, 18 are marked as small S, medium M, or large L.

While the implant 12 is illustrated as a cylinder, as should be apparent to those skilled in the art, the implant 12 is contemplated to be a part of an implant 12, such as a post, for example. The shape, size, and type of implant 12 is not considered to be limiting of the present invention, as made more clear by the discussion presented hereinbelow.

After a suitable adapter 14, 16, 18 is selected by the surgeon, the adapter 14, 16, 18 is inserted into the proximal end of the tibia 10. In particular, the adapter 14, 16, 18 is inserted into the bone marrow channel C at the center of the tibia. Thereafter, the implant 12 is positioned within the adapter 14, 16, 18. The adapter 14, 16, 18 helps to secure the implant in the proximal end of the tibia 10 by improving engagement with the tibia 10, as discussed above.

As also discussed above, there are difficulties associated with the use of adapters 14, 16, 18. Among the difficulties, the adapter 14, 16, 18 may not provide a precise fit in the channel C in the tibia 10.

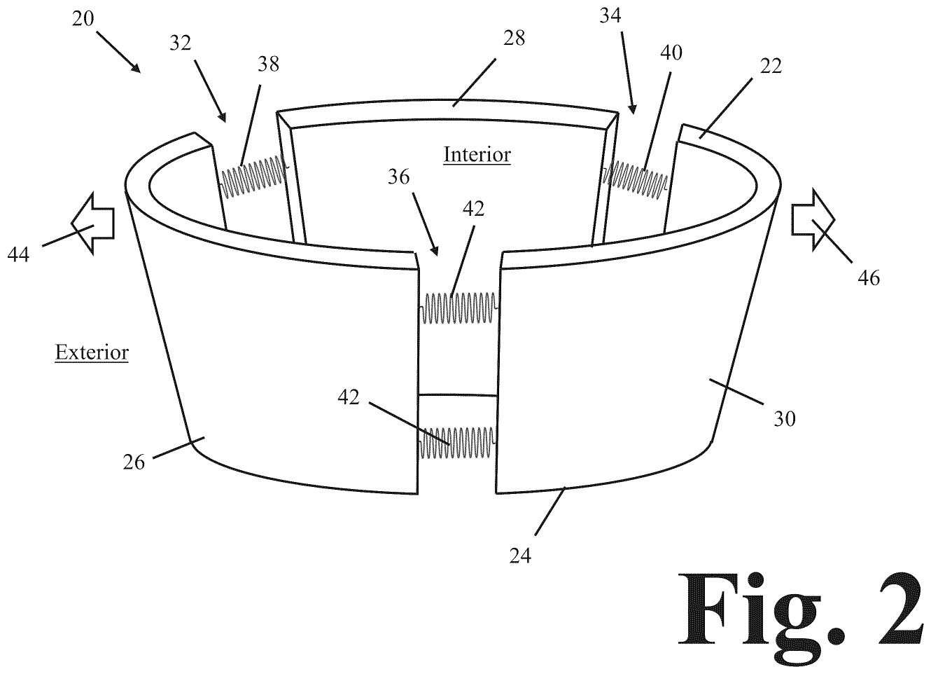

To address one or more of the difficulties associated with the prior art, the present invention provides several embodiments of an adapter, as discussed in connection with FIGS. 2-9.

As will be made apparent from the discussion that follows, one aspect of the present invention is the provision of an adapter that may be inserted into the proximal end of the tibia 10 where the size of the adapter is adjustable. Instead of relying on adapters 14, 16, 18 that are constructed with differing sizes, the size of the adapter of the present invention is adaptable to the size of the channel C. In other words, the size of the adapter is changeable to accommodate the particular circumstances presented by the patient. This includes, but is not limited to, adjusting the adapter to the size of the channel C and also adjusting the adapter to the shape of the channel C.

FIG. 2 is a perspective, graphical illustration of a first embodiment of an adapter 20 according to the present invention.

With reference to FIG. 2, the adapter 20 is frusto-conically shaped. The adapter 20 defines an interior and an exterior, as indicated.

It is contemplated that the frusto-conical shape will be preferred for the majority of implementations, including, but not limited to, knee replacement surgeries.

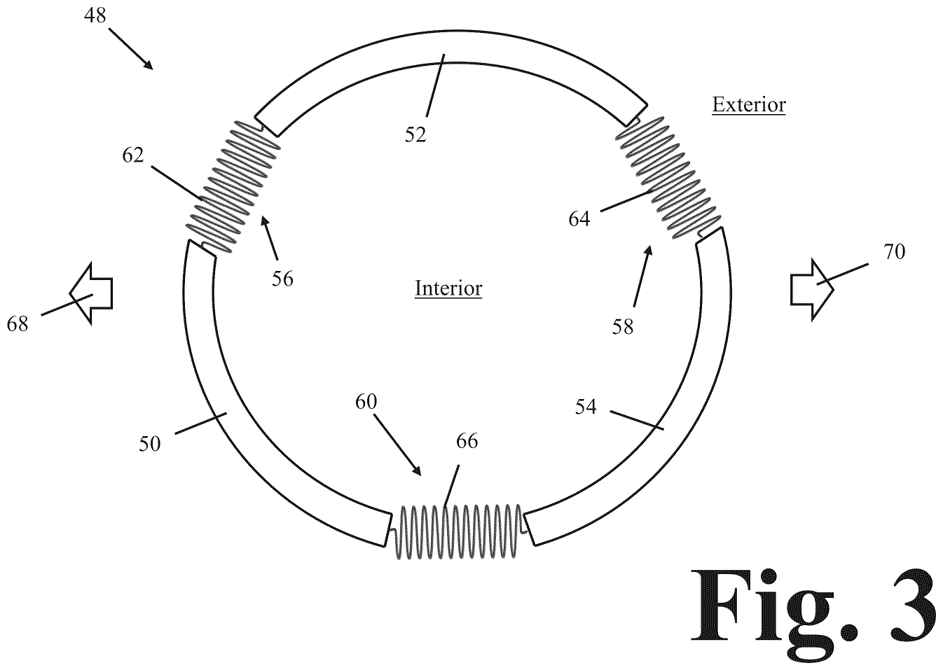

Although the adapter 20 is illustrated with a frusto-conical shape, the present invention should not be understood to be limited to any particular shape and/or configuration. To the contrary, the adapter 20 may take any shape without departing from the scope of the present invention. For example, the adapter 20 may be cylindrically shaped, as shown in FIG. 3, for example. Still further, the adapter 20 may have a non-circular cross-section. For example, the adapter 20 may have an elliptical and/or non-regular cross-section, among others.

The adapter 20 has a proximal side 22 and a distal side 24. The distal side 24 is contemplated to be inserted into the channel C in the proximal end of the tibia 10. It is noted, however, that the designation of a proximal side 22 and a distal side 24 does not limit the present invention. Depending on the circumstances, the proximal side 22 may be the side inserted into the tibia 10, as should be apparent to those skilled in the art.

The adapter 20 includes three rigid segments, a first rigid segment 26, a second rigid segment 28, and a third rigid segment 30. The first rigid segment 26 is separated from the second rigid segment 28 by a first gap 32. The second rigid segment 28 is separated from the third rigid segment 30 by a second gap 34. The third rigid segment 30 is separated from the first rigid segment 26 by a third gap 36.

While three rigid segments 26, 28, 30 are shown, it is noted that any number of rigid segments greater than one may be suitable for the present invention. In a case where one rigid segment is employed, the rigid segment may have a C-shaped configuration.

Still further, it is contemplated that the adapter of the present invention may include two rigid segments. With two rigid segments, the rigid segments may be configured as half circles, for example.

While an adapter with one or two rigid segments is contemplated to fall within the scope of the present invention, one or more embodiments are contemplated to include a plurality of rigid segments 26, 28, 30, as shown.

With continued reference to FIG. 2, the three rigid segments 26, 28, 30 are connected to one another via a first flexible segment 38, a second flexible segment 40, and third flexible segment 42. The flexible segments 38, 40, 42 are illustrated as spring members that push the rigid segments 26, 28, 30 apart from one another. As a result, together, the rigid segments 26, 28, 30 and the flexible segments 38, 40, 42 are constructed to establish a suitable hoop stress that biases the adapter 20 in an expanded state, as indicated by the arrows 44, 46. Together, the rigid segments 26, 28, 30 and the flexible segments 38, 40, 42 provide a sufficient hoop stress so that, when the adapter 20 is inserted into the central channel C in the tibia 10, the adapter 20 exerts sufficient forces, as indicated by the arrows 44, 46, on the interior walls of the channel C to discourage dislodgement of the adapter 20 therefrom. The arrows 44, 46 indicate forces directed from the interior of the adapter 20 to the exterior of the adapter 20.

For clarity, it is contemplated that the hoop stress exerted by the adapter 20 exerts forces around the entire periphery of the adapter 20, not just at the ends indicated by the arrows 44, 46. The hoop stress may be uniform around the periphery of the adapter 20. Alternatively, the hoop stress may be applied at one or more discrete locations around the periphery of the adapter 20. As noted, the hoop stress applies forces in a direction exteriorly to the adapter 20.

With continued reference to FIG. 2, the rigid segments 26, 28, 30 are contemplated to be made from a material that encourages growth of bone thereinto. For example, the rigid segments 26, 28, 30 may be made from sintered titanium, porous metal alloys, porous ceramic materials, polymers, and/or other biocompatible materials that are familiar to those skilled in the art.

It is also contemplated that the exterior surfaces of the rigid segments 26, 28, 30 will present a roughened surface to the interior surface of the channel C. With a roughened surface, it is contemplated that the rigid segments 26, 28, 30 will present a frictional engagement that, together with the hoop stress generated by the adapter 20, initially secures the adapter 20 in the proximal end of the tibia 10.

After the passage of time, bone will grow into the porous structure of the rigid segments 26, 28, 30 to adhere more fully the adapter to the tibia 10.

Since the details of bone tissue growth and regeneration are known to those skilled in the art, further details are not provided here. Simply, the materials used to construct one or more segments of the adapter 20 will permit bone growth thereinto, thereby establishing a secure fusion between the adapter 20 and the tibia 10.

The flexible segments 38, 40, 42 are illustrated as spring-like elements in FIG. 2. While the flexible segments 38, 40, 42 may be configured as springs, such a configuration is not considered to be limiting of the present invention. The flexible segments 26, 28, 30 may have any construction without departing from the scope of the present invention.

As noted above, the flexible segments 38, 30, 42 are contemplated to push the rigid segments 26, 28, 30 away from one another to establish a sufficient hoop stress to secure the adapter 20 within the proximal end of the tibia 20.

In FIG. 2, two flexible elements 38, 40, 42 are contemplated to be positioned in each of the gaps 32, 34, 36. However, this configuration is merely exemplary.

It is contemplated that only one flexible segment 38, 40, 42 may be positioned between each rigid elements 26, 28, 30. Still further, it is contemplated that two or more flexible segments 38, 40, 42 may be positioned between the rigid elements 38, 40, 42. The exact number of flexible segments 38, 40, 42, is contemplated to depend on various factors including, but not limited to, the size of the adapter 20, the hoop stress required, the materials employed to construct the flexible segments 38, 40, 42, etc. Any number of flexible segments 38, 40, 42 greater than one between adjacent rigid segments 26, 28, 30 is considered to be suitable for the adapter 20 of the present invention.

The flexible segments 38, 40, 42 are contemplated to be made from any suitable material including, but not limited to, titanium, metal alloys, shape metal alloys (“SMAs”), ceramics, polymers, biocompatible materials, and the like. The exact material employed for the flexible segments 38, 40, 42 should not be understood to limit the scope of the present invention.

FIG. 3 is a graphical, top view of a second embodiment of an adapter 48 according to the present invention. The adapter 48 defines an interior and an exterior, as indicated.

The adapter 48 shares much of the same construction as the adapter 20 illustrated in FIG. 2. Here, however, the rigid segments 50, 52, 54 do not form a frusto-conical shape. Instead, the rigid segments 50, 52, 54 establish a cylindrical shape that may be inserted into the channel C in the proximal end of the tibia 10.

The rigid segments 50, 52, 54 are contemplated to be constructed from the same materials described in connection with the rigid segments 26, 28, 30 described in connection with the adapter 20.

The rigid segments 50, 52, 54 are separated from one another by gaps 56, 58, 60.

The rigid segments 50, 52, 54 are connected to one another via a first flexible segment 62, a second flexible segment 64, and a third flexible segment 66, just as for the adapter 20. The flexible segments 62, 64, 66 are contemplated to have the same arrangement and distribution as discussed in connection with the adapter 20.

As before, the flexible segments 62, 64, 66 bias the rigid segments 50, 52, 54 outwardly (or exteriorly), as indicated by the arrows 68, 70. Together, the rigid segments 50, 52, 54 and the flexible segments 62, 64, 66 are contemplated to generate a sufficient hoop stress to hold the adapter 48 in place after being inserted into the tibia 10.

As before, the hoop stress is indicated by the arrows 68, 70. As with the adapter 20, the forces indicated by the arrows 68, 70 are contemplated to bias the adapter 48 into an expanded state.

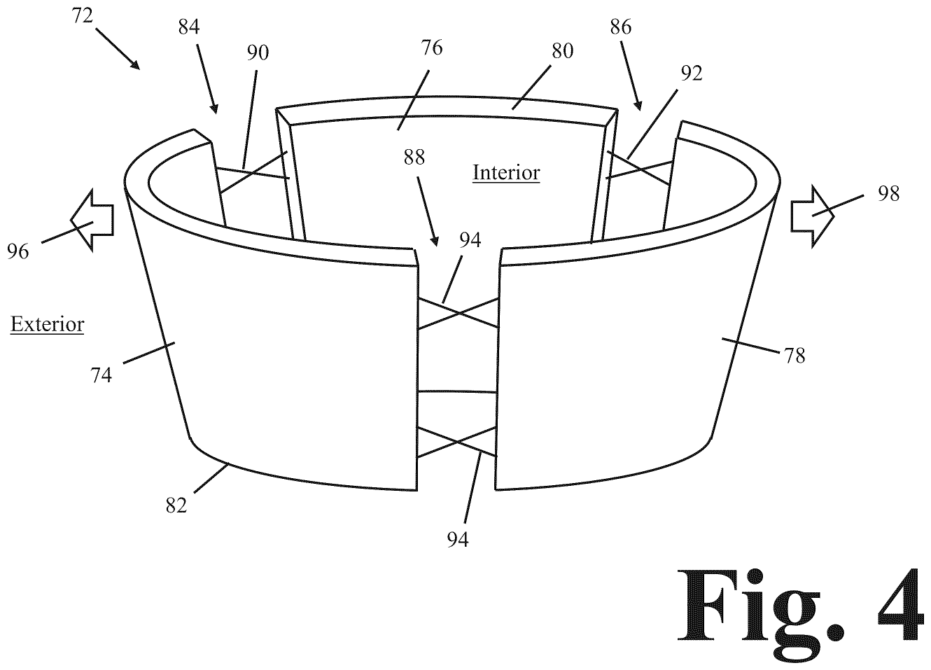

FIG. 4 is a graphical, perspective illustration of a third embodiment of an adapter 72 according to the present invention. The adapter 72 defines an interior and an exterior.

The adapter 72 shares much of the same construction as the adapters 20, 48 illustrated in FIGS. 2 and 3. In this embodiment, the rigid segments 74, 76, 78 form a frusto-conical shape like the adapter 20. As before, the adapter 72 has a proximal side 80 and a distal side 82.

The rigid segments 74, 76, 78 are contemplated to be constructed from the same materials described in connection with the rigid segments 26, 28, 30 described in connection with the adapter 20.

The rigid segments 74, 76, 78 are separated from one another by first, second, and third gaps 84, 86, 88.

The rigid segments 74, 76, 78 are connected to one another via a first flexible segment 90, a second flexible segment 92, and a third flexible segment 94, just as for the adapters 20, 48.

The flexible segments 90, 92, 94 are contemplated to have a construction that differs from the construction of the flexible segments described in connection with the adapters 20, 48. Here, the flexible segments 90, 92, 94 are contemplated to have a scissor or scissor-like construction that biases the rigid segments 74, 76, 78 outwardly, as indicated by the arrows 96, 98. As before, the rigid segments 74, 76, 78 and the flexible segments 90, 92, 94 are contemplated to generate a sufficient hoop stress to hold the adapter 72 in place after being inserted into the tibia 10.

As before, the hoop stress is indicated by the arrows 96, 98. As with the adapters 20, 48, the forces indicated by the arrows 96, 98 are contemplated to bias the adapter 72 into an expanded state.

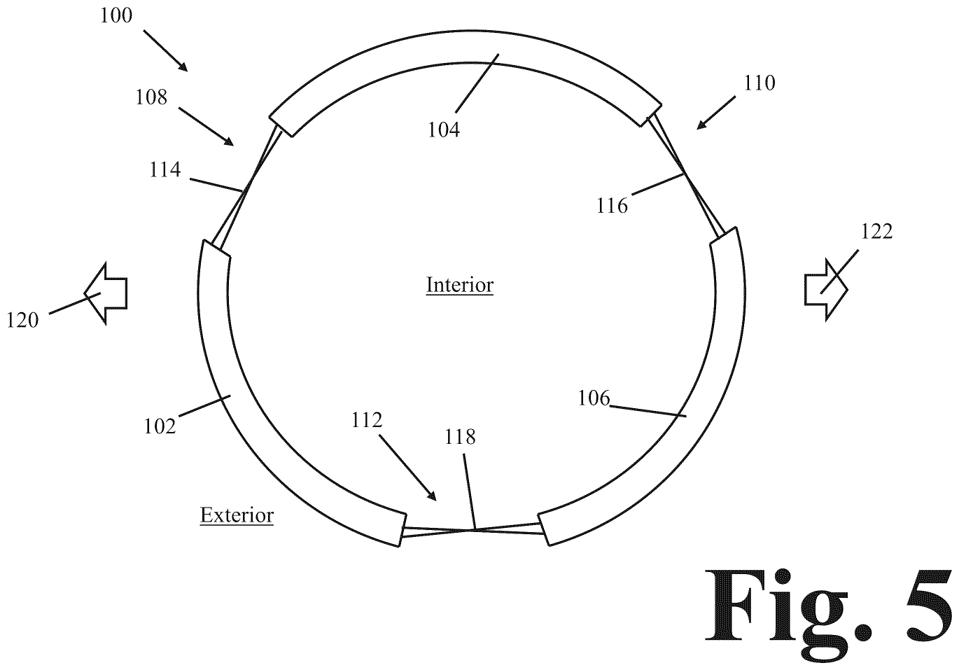

FIG. 5 is a graphical, top view of a fourth embodiment of an adapter 100 according to the present invention. The adapter 100 defines an interior and an exterior.

The adapter 100 combines elements of the adapter 72 illustrated in FIG. 4 with the adapter 48 illustrated in FIG. 3. In this embodiment, as with the adapter 48, the rigid segments 102, 104, 106 do not form a frusto-conical shape. As with the adapter 48, the rigid segments 102, 104, 106 establish a cylindrical shape that may be inserted into the channel C in the proximal end of the tibia 10.

The rigid segments 102, 104, 106 are contemplated to be constructed from the same materials described in connection with the rigid segments 26, 28, 30 described in connection with the adapter 20 and the rigid segments 50, 52, 54 discussed in connection with the adapter 48.

The rigid segments 102, 104, 106 are separated from one another by gaps 108, 110, 112.

The rigid segments 102, 104, 106 are connected to one another via a first flexible segment 114, a second flexible segment 116, and a third flexible segment 118. The flexible segments 114, 116, 118 are contemplated to be scissor or scissor-like elements, as described in connection with the adapter 72 shown in FIG. 4.

As before, the flexible segments 114, 116, 118 bias the rigid segments 102, 104, 106 outwardly, as indicated by the arrows 120, 122. Together, the rigid segments 102, 104, 106 and the flexible segments 114, 116, 118 are contemplated to generate a sufficient hoop stress to hold the adapter 100 in place after being inserted into the tibia 10.

As before, the hoop stress is indicated by the arrows 120, 122.

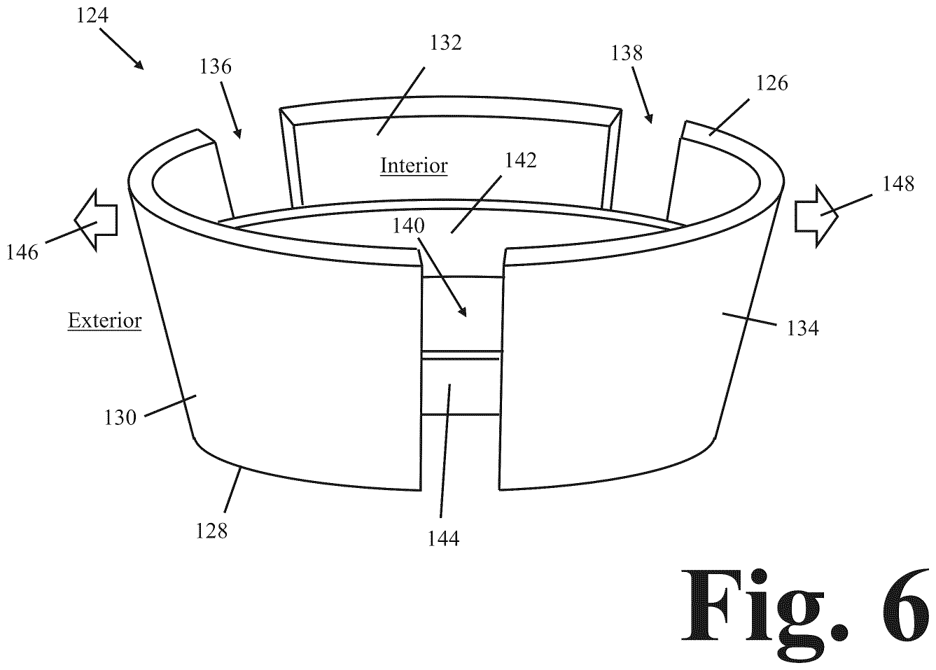

FIG. 6 is a graphical, perspective illustration of a fifth embodiment of an adapter 124 according to the present invention. The adapter 124 defines an interior and an exterior.

As in other embodiments, the adapter 124 has a proximal side 126 and a distal side 128.

The adapter 124 also includes a first rigid segment 130, a second rigid segment 132, and a third rigid segment 134. The rigid segments 130, 132, 134 are separated by a first gap 136, a second gap 138, and a third gap 140.

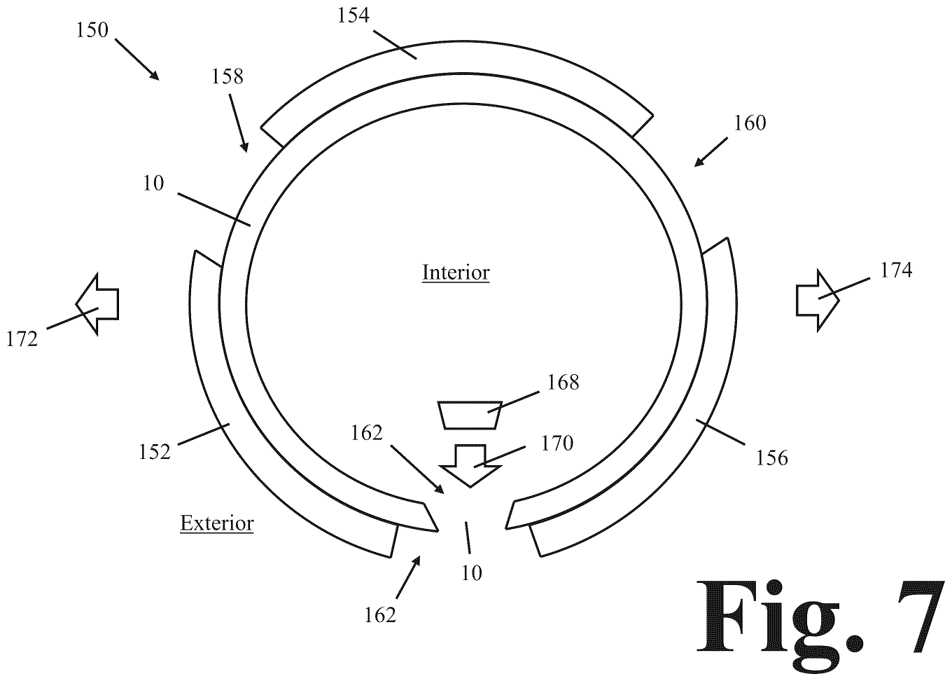

The adapter 124 incorporates a flexible segment 142 in the form of a C-shaped ring that is connected to the rigid segments 130, 132, 134 on the interior surfaces thereof. As more clearly illustrated in FIG. 7, the flexible segment 142 optionally may include a key 144 to establish the appropriate hoop stress indicated by the arrows 146, 148.

The rigid segments 130, 132, 134 are contemplated to be made from materials discussed in connection with other embodiments.

The flexible segment 142 is contemplated to be constructed from one or more materials discussed in connection with other embodiments as well.

The flexible segment 142 exerts an outward force on the rigid segments 130, 132, 134 to push the rigid segments 130, 132, 134 outward in the direction of the arrows 146, 148. Together, the interaction between the flexible segment 142 and the rigid segments 130, 132, 134 establish a sufficient hoop stress to retain the adapter 124 in the proximal end of the tibia 10.

It is noted that the key 144 is contemplated to be an element that may be inserted into the flexible segment 142, details of which are mor fully discussed in connection with FIG. 7. The key 144 completes the ring shape of the flexible segment 142 and, therefore, is contemplated to enhance the hoop stress generated by the flexible segment 142. The key 144 is contemplated to be optional, however, because the flexible segment 142 should be understood to generate sufficient hoop stress without the key 144.

FIG. 7 is a graphical, top view of a sixth embodiment of an adapter 150 according to the present invention. The adapter 150 defines an interior and an exterior.

The adapter 150 is similar to the adapter 124 illustrated in FIG. 6, except that the rigid segments 152, 154, 156 form a cylindrical shape rather than an frusto-conical shape. As before the rigid segments 152, 154 156 are separated by gaps 158, 160, 162.

As with the adapter 124, the adapter 150 includes a flexible segment 164 that is C-shaped. The flexible segment 164 is disposed interiorly to the rigid segments 152, 154, 156 to which the flexible segment 164 is connected. The flexible segment 164 is contemplated to exert sufficient hoop stress to secure the adapter 150 in the proximal end of the patient's tibia 10.

The flexible segment 164 includes an opening or keyway 166 therein. Optionally, a key 168 may be inserted into the opening 166 to complete the flexible segment 164. If inserted, the key 168 may be positioned in the direction of the arrow 170.

It is contemplated that the flexible segment 164 will generate sufficient hoop stress as indicated by the arrows 172, 174.

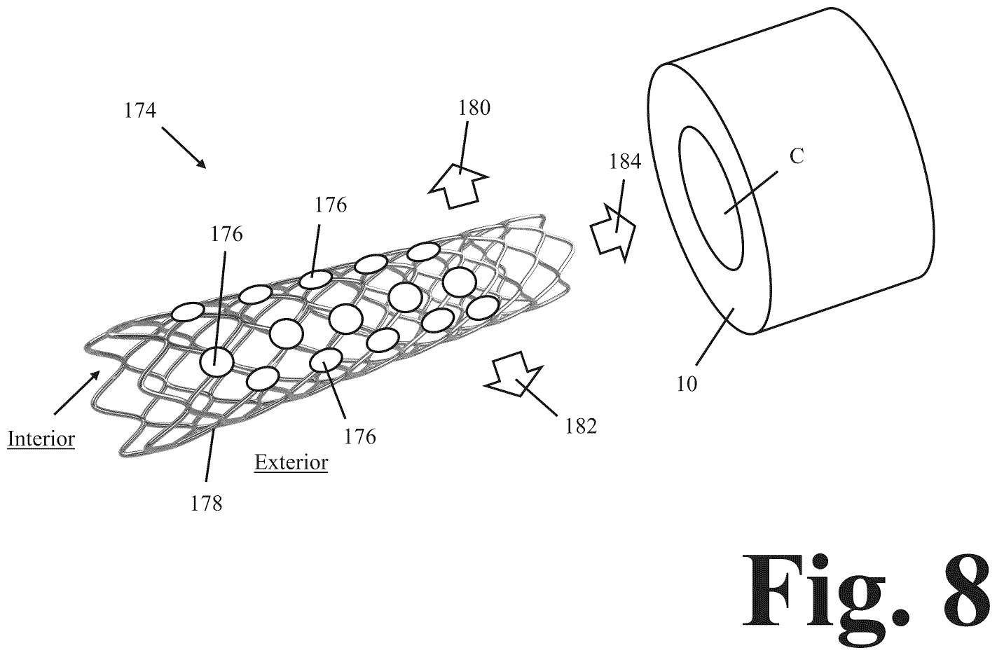

FIG. 8 is a graphical, perspective illustration of a seventh embodiment of an adapter 176 according to the present invention. The adapter 176 defines an interior and an exterior.

In this embodiment, the adapter 174 includes rigid segments 176, shown as circular pads, disposed on a flexible segment 178, shown as a mesh. The rigid segments 176 are contemplated to be made from one or more of the materials discussed hereinabove. Similarly, the flexible segment 178 is contemplated to be made from one or more of the materials discussed herein.

As before, the rigid segments 176 and the flexible segment 178 are contemplated to cooperate to generate an outwardly biased hoop stress indicated by the arrows 180, 182. As before, the adapter 174 is contemplated to be inserted into the proximal end of a tibia 10 as indicated by the arrow 184.

While the adapter 174 is illustrated as having a frusto-conical shape, it is noted that the adapter 174 may have any other shape without departing from the scope of the present invention. For example, without limiting the present invention, the adapter 174 may be cylindrically shaped.

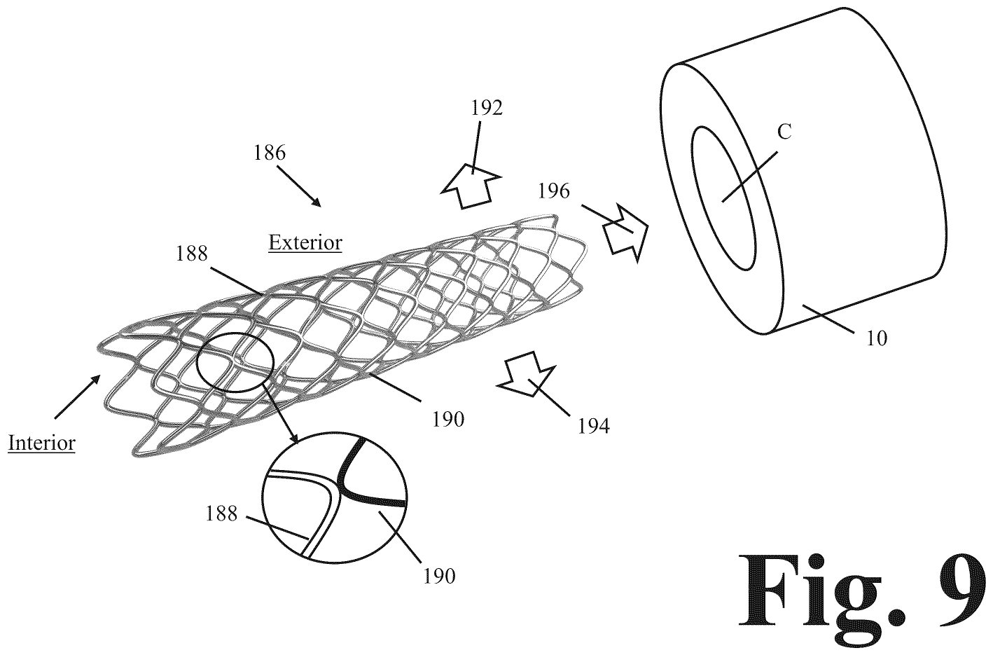

FIG. 9 is a graphical, perspective illustration of an eighth embodiment of an adapter 186 according to the present invention. The adapter 186 defines an interior and an exterior.

The adapter 186 is contemplated to be similar to the adapter 174 illustrated in FIG. 8. The adapter 186 incorporates the rigid segments 188 into the flexible segments 190, forming the mesh. Here, for example, it is contemplated that the rigid segments 188 and the flexible segments 190 are combined to form the mesh structure of the adapter 186, as indicated in the enlarged bubble provided in FIG. 9.

As before, it is contemplated that the rigid segments 188 and the flexible segments 190 will cooperate to establish a sufficient hoop stress, indicated by the arrows 192, 194, to retain the adapter 186 within the channel C in the patient's tibia. The arrow 196 indicates the direction of insertion of the adapter 186 into the patient's tibia 10.

While the adapter 186 is illustrated as having a frusto-conical shape, it is noted that the adapter 186 may have any other shape without departing from the scope of the present invention. For example, without limiting the present invention, the adapter 186 may be cylindrically shaped.

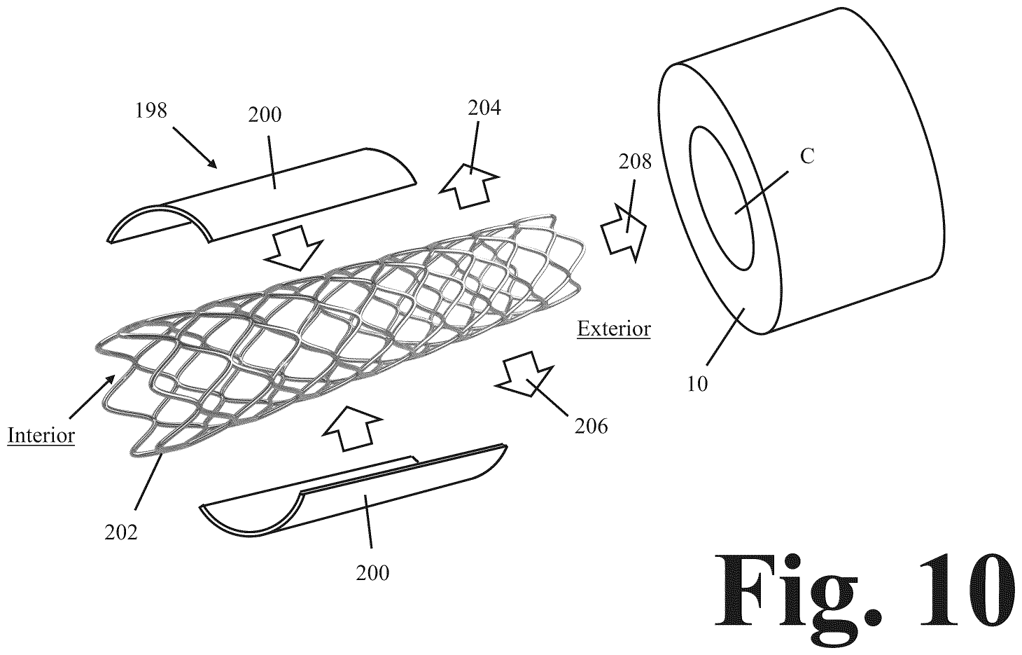

FIG. 10 is a graphical, perspective illustration of a ninth embodiment of an adapter 198 according to the present invention. The adapter 198 defines an interior and an exterior.

In this embodiment, the adapter 198 includes rigid segments 200, shown as partial frusto-conical and/or partial cylindrical segments, disposed on a flexible segment 202, shown as a mesh. The rigid segments 200 are contemplated to be made from one or more of the materials discussed hereinabove. Similarly, the flexible segment 202 is contemplated to be made from one or more of the materials discussed herein.

As before, the rigid segments 200 and the flexible segment 202 are contemplated to cooperate to generate an outwardly biased hoop stress indicated by the arrows 204, 206. As before, the adapter 198 is contemplated to be inserted into the proximal end of a tibia 10 as indicated by the arrow 208.

While the adapter 198 is illustrated as having a frusto-conical shape, it is noted that the adapter 198 may have any other shape without departing from the scope of the present invention. For example, without limiting the present invention, the adapter 198 may be cylindrically shaped.

For clarity, it is noted that the combination of the rigid segments and the flexible segments for the various embodiments of the adapter 20, 48, 72, 100, 124, 150, 174, 186, 198 of the present invention together form a “body.” The body is inserted into the patient's tibia 10, as discussed hereinabove.

Also for clarity, it is noted that the adapter 20, 48, 72, 100, 124, 150, 174, 186, 198 of the present invention should not be understood to be employed only in a tibia for knee replacement surgery. As should be apparent to those skilled in the art, the adapter 20, 48, 72, 100, 124, 150, 174, 186, 198 of the present invention may be employed in any bone for any suitable surgical purpose. In particular, the adapter 20, 48, 72, 100, 124, 150, 174, 186, 198 is contemplated to be employed in long bones in the human body, because long bones present a channel into which the adapter 20, 48, 72, 100, 124, 150, 174, 186, 198 may be readily inserted. Without limiting the present invention, the femur and arm bones are suitable candidates for the adapter 20, 48, 72, 100, 124, 150, 174, 186, 198.

The adapter 20, 48, 72, 100, 124, 150, 174, 186, 198 should not be understood to be limited to use on humans. The adapter 20, 48, 72, 100, 124, 150, 174, 186, 198 may be employed in any vertebrate without departing from the scope of the present invention.

The adapter 20, 48, 72, 100, 124, 150, 174, 186, 198 is provided so that an implant 12 may be inserted thereinto. The implant 12 may be a replacement knee, for example, as indicated above. As should be apparent, however, any other implant may be inserted into the adapter 20, 48, 72, 100, 124, 150, 174, 186, 198 without departing from the scope of the present invention.

As discussed hereinabove, the embodiments of the present invention are exemplary only and are not intended to limit the present invention. Features from one embodiment are interchangeable with other embodiments, as should be apparent to those skilled in the art. As such. variations and equivalents of the embodiments described herein are intended to fall within the scope of the claims appended hereto.

What is claimed is:

1. An adapter for receiving an implant, comprising:
at least one rigid segment; and
at least one flexible segment connected to the at least one rigid segment,
wherein the at least one rigid segment and the at least one flexible segment together form a body,
wherein the at least one rigid segment comprises a material encouraging growth of bone thereinto,
wherein the body defines an interior and an exterior,
wherein the at least one rigid segment and the at least one flexible segment bias the body to expand outwardly from the interior,
wherein the body is adapted to be inserted into a bone.

2. The adapter according to claim 1, wherein the at least one rigid segment comprises at least one of sintered titanium, a porous metal alloy, a porous ceramic material, a polymer, and a biocompatible material.

3. The adapter according to claim 1, wherein the at least one flexible segment comprises titanium, a metal alloy, a shape metal alloy, a ceramic, a polymer, and a biocompatible material.

4. The adapter according to claim 1, wherein the body is frusto-conically shaped.

5. The adapter according to claim 1, wherein the body is cylindrically shaped.

6. The adapter according to claim 1, wherein the body defines a mesh.

7. The adapter according to claim 1, wherein the at least one flexible segment is a spring.

8. The adapter according to claim 1, wherein the at least one flexible segment is a scissor element.

9. The adapter according to claim 1, wherein the at least one flexible segment is C-shaped.

10. The adapter according to claim 1, wherein the at least one rigid segment comprises at least two rigid segments separated from one another by gaps.

11. The adapter according to claim 10, wherein the at least one flexible segment comprises a plurality of flexible segments disposed in the gaps, connecting the at least two rigid segments together.

12. The adapter according to claim 9, wherein the C-shaped flexible segment defines a keyway, the adapter further comprising:
a key insertable into the keyway.

13. The adapter according to claim 6, wherein the at least one flexible segment forms the mesh and the at least one rigid segment is disposed on the at least one flexible segment.

14. The adapter according to claim 6, wherein the at least one rigid segment and the at least one flexible segment together form the mesh.
